(19) United States
(12) Patent Application Publication
(10) Pub. No.: US 2008/0299624 A1
(43) Pub. Date: Dec. 4, 2008

(54) Continuous fermentation apparatus and method

(76) Inventor: Edward Heslop, Aurora, NY (US)

Correspondence Address: SIMPSON & SIMPSON, PLLC, 5555 MAIN STREET, WILLIAMSVILLE, NY, 14221-5406, US

(21) Appl. No.: 11/809,771

(22) Filed: Jun. 1, 2007

(51) Int. Cl. C12M 3/00; C12P 1/04; C12P 17/18; C12P 7/14; C12P 7/22

(52) U.S. Cl. 435/119; 435/156; 435/162; 435/170; 435/303.1

(57) ABSTRACT

The present invention includes a method for the continuous fermentation of a desired product by the establishment of a steady state condition in a reactor followed by the nonsequential discharge of nutrient media through a plurality of intake ports. The nonsequential discharge of a plug of nutrient media causes a localized segment of the fermentation microorganisms to mix with the media while the remainder of the bed of microorganisms remains quiescent. An apparatus for practicing the method is also disclosed.

### FIELD OF THE INVENTION

The present invention pertains to fermentation methods and apparatus, more specifically to fermentation systems incorporating the continuous removal of fermentation products and more specifically, the use of multiple growth media input ports.

### BACKGROUND OF THE INVENTION

Through most of recorded history, fermentation methods and apparatus have been utilized to produce organic products through the enzymatic breakdown of complex organic precursors into simple final products such as alcohol and acetic acid. One common problem faced in these operations is that of batch production methodology, in which production of a desired product is generated by a fermentation method which functions for a defined period of time to produce a finite amount of final product. Normally, production naturally stops due to the inactivation of the microorganism by increased product concentrations. After production of the final product, the operation must be restarted with a consequent loss of production time. This downtime can be exacerbated if the production equipment has to be cleaned to allow production of a different product.

Two of the factors that encumber continuous fermentation methods are the potential for inadequate exposure of the fermentation organism to nutrient media and the need for a quiescent period during which the microorganism has time to interact with newly introduced nutrient media and waste material can be removed. The quiescent period not only allows for more efficient metabolic activity in the organism but also facilitates the creation of a product gradient to allow for more economical removal of the product and waste.

However, conditions needed for adequate exposure to nutrient media are often directly opposite to what is needed to create quiescent conditions for adequate media interaction with the fermentation microorganisms and efficient product output by the fermentation microorganisms. Exposure to nutrient media most often requires the exposure of microorganisms to newly introduced nutrient media by mixing. Movement of microorganisms generated by mixing with nutrient media disrupts the quiescent period during which product output is most efficient. In addition, prolonged quiescent periods will eventually lead to a slowdown of product output by the microorganism caused by increased concentration of product and the buildup of metabolic waste around the cells. Without mixing to remove the build up of product and waste, the microorganisms will be destroyed or at least crippled.

An additional factor that hampers continuous fermentation processes is the problem of loss of microorganisms. As microorganisms are mixed and product is drawn off, microorganisms are often removed from the reactor with the output stream and are either lost or must be recirculated back into the reactor. Such movement can lead to the deterioration of the microorganism bed and requires eventual shut down to remove the exhausted microorganism bed. For example, U.S. Pat. No. 4,952,503 to Granstedt discloses an ethanol production process in which fermentation liquid is continuously withdrawn from a reactor in which the liquid is first strained and then separated from the yeast. Among the remaining steps, the yeast is recirculated into the reactor. U.S. Pat. No. 4,358,536 to Thorsson, et al. discloses a fermentation method in which both a yeast concentrate flow stream and a yeast-free flow stream are separated out of a fermentation liquor with the liquid portion of the yeast free flow stream recirculated back into the reactor along with the yeast concentrate flow. However, this system requires that the fermentation microorganisms, namely yeast, be continuously recirculating out of and back into the reactor which leads to deterioration of the yeast bed.

### SUMMARY OF THE INVENTION

The present invention broadly comprises a process for the continuous production of a fermentation product comprising establishing a steady state condition for a colony of a fermentation microorganism on a bed of support particles in a production reactor, discharging a quantity of nutrient medium into the production reactor sequentially one at a time through at least two nonadjacent intake ports of a plurality of discharge ports to mix the nutrient medium with a segment of the colony, allowing the segment to settle into the bed of support particles, separating the fermentation product from the nutrient medium, withdrawing the nutrient medium from the production reactor, recirculating the nutrient medium into the fermentation colony, and repeating the sequence from the nutrient discharge step to the step of recirculating the nutrient medium into the fermentation colony.

The present invention also includes an apparatus for performing the continuous fermentation method broadly comprising a fermentation reactor having a colony of a fermentation microorganisms attached to a bed of support particles, a nutrient medium tank, a plurality of nutrient medium discharge pipes operatively connected between the fermentation reactor and the nutrient medium tank, an input pump operatively arranged to pump the nutrient medium between the nutrient medium tank and the fermentation reactor through the plurality of nutrient medium discharge pipes, a sequential controller operatively connected between the plurality of the nutrient medium discharge pipes and the input pump for controlling the sequence or order in which nutrient medium is input through each of the plurality of nutrient medium intake ports (discharge ports), and a fermentation product tank connected to the fermentation reactor and arranged to receive the fermentation product. By operatively connected is meant that one component is positioned between two other components so as to perform or control a particular operation(s) affecting the two other components, even if there is not a direct connection between the components.

In addition, the present invention also includes a fermentation product made by a process broadly comprising establishing a steady state condition for a colony of a fermentation microorganisms on a bed of support particles in a production reactor, discharging a quantity of nutrient medium into the production reactor sequentially one at a time through at least two nonadjacent intake ports of a plurality of intake ports thereby mixing the nutrient medium with a segment of the colony, allowing the mixed segment to settle into the bed of support particles, separating the fermentation product from the nutrient medium, withdrawing the nutrient medium from the production reactor, recirculating the nutrient medium into the fermentation colony and, repeating the above steps of discharging nutrient medium into the reactor through recirculating the withdrawn nutrient medium into the reactor.

An object of the invention is to provide a method of maintaining a fermentation reaction without the need to stop and restart the reaction.

A second object of the invention is to establish a method of mixing nutrient media in a bed of fermentation microorganisms while maintaining a generally quiescent bed.

A third object of the invention is to disclose an apparatus constructed to enable mixing of nutrient media in a segment of a bed of fermentation microorganisms while maintaining a generally quiescent state throughout the greater portion of the bed.

An additional object of the invention is to make known a method of controlling the segment of a bed that receives nutrient media.

A further object of the invention is to disclose one or more fermentation products made by the disclosed method.

### BRIEF DESCRIPTION OF THE SEVERAL VIEWS OF THE DRAWINGS

The nature and mode of the operation of the present invention will now be more fully described in the following detailed description of the invention taken with the accompanying drawing Figures, in which

FIG. 1 is a schematic diagram of the continuous fermentation apparatus of the present invention;

FIG. 1A is a schematic diagram of an alternate embodiment of the present invention in which the direction of flow into the reactor is reversed;

FIG. 2 is a graph showing the levels of various production parameters throughout a production run;

FIG. 2A is a graph extracting from FIG. 2 the quantities of ethanol and inositol produced during the production run;

FIG. 3 depicts the level of dissolved oxygen measured throughout the production run;

FIG. 4 depicts the lack of temperature variation throughout the duration of the production run; and,

FIG. 5 shows the pH range throughout the production run.

### DETAILED DESCRIPTION OF THE INVENTION

At the outset, it should be appreciated that like drawing numbers on different drawing views identify identical structural elements of the invention.

While the present invention is described with respect to what is presently considered to be the preferred embodiments, it is understood that the invention is not limited to the disclosed embodiments. The present invention is intended to cover various modifications and equivalent arrangements included within the spirit and scope of the appended claims.

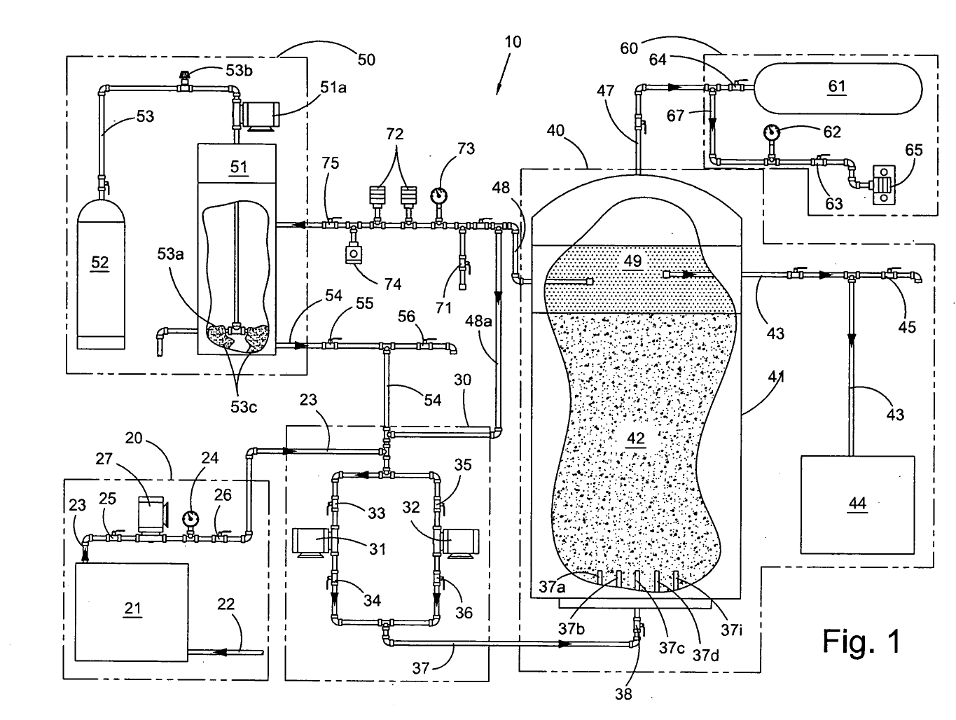

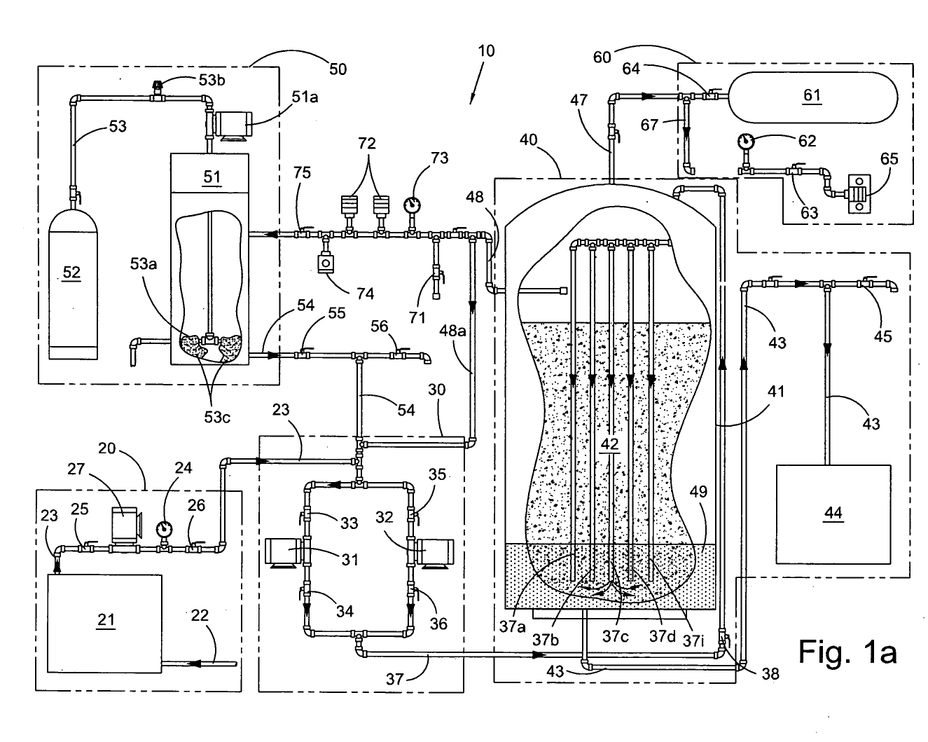

Adverting to the drawings, FIG. 1 is a schematic diagram of continuous fermentation system 10 (“system 10”) of the present invention. System 10 comprises a series of connected subsystems that together enable the continuous production of fermentation products.

Media input system 20 includes influent broth tank 21 (“feed tank 21”) which receives growth media from the system operator through input line 22. Parameters used to determine the composition feed media include, but are not limited to, target product to be produced, collected, and stored, fermentation microorganism (“microorganism”) being utilized, and particles used to support the microorganism. As will be explained below, nutrient media from feed tank 21 is delivered to microorganism bed 42 in reactor 41 through a plurality of input pipes. Because the maximum delivery rate can be increased or decreased by the number of input pipes 37, the input rate and capacity of system 10 can range from small bench continuous systems to multi-million gallon capacity systems. Depending on the parameters listed above, as well as the desired production capacity of system 10, feed tank 21 may be fabricated to be of any suitable size, shape, and material necessary to meet production goals.

Outlet pipe 23 leaves feed tank 21 and leads to recirculation system 30. In one embodiment, outlet pipe 23 is fabricated from polypropylene, but other materials such as stainless steel, glass or polyethylene may be used as long as the fabrication material can be sterilized or sanitized to remove undesired microorganisms. Pump 27 is used to pump the feed media from feed tank 21 to recirculation system 30 or optionally directly to reactor 41. In this sense, pump 27 is operatively connected to reactor 41 meaning that nutrient media pumped by pump 27 is connected to reactor 41 either directly or indirectly through one or more recirculation systems 30. In a preferred embodiment, valves 25 and 26 are placed upstream and downstream, respectively, from pump 27 to allow the flow of feed media to be shutoff if necessary. One type of valve can be a ⅜inch ball valve which may be hand operated or mechanically or electronically operated. In a still more preferred embodiment, pressure gauge 24 is used to indicate pump flow pressure from pump 27.

Pipe 23 intersects with recirculation system 30 through aeration outlet pipe 54 which extends from aeration chamber 51. Recirculation system 30 includes bump pump 31 and recycle pump 32. A timer (not shown) is operatively connected to bump pump 31 to control the pump cycle of bump pump 31 as described below. Recirculation pump 32 maintains pressure and movement of the feed media and, optionally water, needed to pulse the biomass bed 42 of fermentation microorganisms in growth reactor 41 (“reactor 41”) to either lift the bed or push down the bed to create the necessary mixing of the feed media around the microorganisms of the biomass 42. Recirculation system 30 eliminates the need to input excess nutrient to maintain system pressure. In addition, recirculation system 30 enables the introduction of new or additional material, such as oxygen, pH adjustment compounds, and nutrients, as needed. In a preferred embodiment, valves 33-36 are incorporated into recirculation system 30 to control the amount of fluid flow and pressure within the system.

Pipe 37 leads from recirculation system 30 to growth reactor system 40 (“growth system 40”). Valve 38 is positioned between the recirculation loop 30 and intake ports 37 to control flow to reactor 41. In one embodiment seen in FIG. 1, reactor tank 41 includes a plurality of intake ports 37a-37i that open into the bottom of reactor tank 41. (“i” indicates the total number of intake ports suitable for a particular reactor 41 and will vary in number depending on, among other factors, the size and configuration of reactor 41. In an alternate preferred embodiment seen in FIG. 1a, input pipes 37a-37i may extend into reactor tank 41 from above. Products with a specific gravity heavier than the growth media will gravitate toward the bottom of reactor tank 41 which will usually require that the feed intakes extend toward the bottom of reactor tank 41 from which the output pipe(s) 43 will lead. During input of the feed media, the media is forcefully ejected out one of input pipes 37a-37i against the bottom or floor of reactor 41 and then up into microorganism bed 42 to thoroughly mix the plug of media with the local portion of bed 42. Conversely, if the desired product is lighter than the feed media, it will tend to float toward the top of reactor tank 41 from which it is drawn off through output pipe(s) 43. Fermentation microorganisms may be genetically modified to produce specific product(s) by methods well known to those skilled in the art.

Product is collected in product tank 44 which is connected to reactor tank 41 by means of outlet pipe 43. In one embodiment, product tank 44 is fabricated from stainless steel and includes a cover. However, depending on the product being recovered and the antiseptic conditions required, product tank 44 may be fabricated from other suitable materials such as polyethylene. In a preferred embodiment, sample valve 45 will extend from outlet pipe 43 to allow a user to obtain samples of newly produced product for quality checks.

Nutrient media outlet 48 (“outlet 48”) extends from reactor tank 41. In FIG. 1, outlet 48 is shown extending from the top of reactor tank 41. However, it may also extend from closer to the bottom of reactor tank 41 if the product(s) produced in reactor tank 41 have a specific gravity heavier than the nutrient media. In either case, the product and nutrient media will move toward product layer 49 and separate during this movement. Outlet 48 may directly connect to recirculation system 30 or may connect downstream to auxiliary equipment. In FIG. 1, outlet 48 is shown connected to aeration system 50 and directly connected to recirculation loop 30 through pipe 48a. Also seen are sample valve 71, measurement probes such as conductivity probe 73, pH and temperature probes 72, and dissolved oxygen (DO) probe 74. It will be recognized by those of ordinary skill in the art that other suitable probes may be inserted into the nutrient flow in outlet 48. Measurements from such test probes can be used to determine if the nutrient media is within desired quality parameters and/or if the composition of nutrient feedstock introduced through influent system 20 needs to be adjusted.

An example of auxiliary equipment connected to outlet 48 is aeration system 50. Aeration chamber 51 is connected to outlet 48 through which it receives nutrient media removed from product layer 49. Aeration chamber 51 receives oxygen from oxygen tank 52 through oxygen input 53. Preferably, oxygen input 53 extends into chamber 51 to aeration stones 53a. Oxygen or other gas flow passing through aeration stones 53a is converted into bubbles which flow up into the nutrient media received through outlet 48 to enrich the nutrient media with oxygen, air or other desired fluid material. Nutrient media flows from aeration chamber 51 through aeration outlet 54 and connects to recirculation system 30 or, optionally, to other intermediate systems. This enables the input of other adjustment media to regulate pH, DO, glucose, etc. Valve 55 and sample valve 56 are used to regulate flow, test nutrient quality, and divert metabolic waste (not shown).

Preferably, system 10 is operated on a continuous basis meaning that system 10 functions to maintain the fermentation process in a continual steady state condition. By steady state is meant a condition that changes only within desired parametric ranges during operation. To attain and maintain this steady state condition, two problems must be overcome. First, the fermentation microorganism bed 42 must be maintained in a controlled condition in which the microorganisms are healthy and productive. This requires that the microorganisms be mixed or stirred with fresh nutrient media Second, the desired product (e.g. ethanol, insulin) must be steadily removed to avoid shutdown of the microorganisms due to an over concentration of the product. This steady removal is best accomplished with a quiescent microorganism bed so that microorganisms, their support particles (if any), and nutrient media are not mixed into the desired product.

System 10 is designed to address these problems to enable the desired fermentation process to operate continuously over an indefinite period. As will be explained below, fermentation product is continuously removed from product layer 49 in reactor 41 to effluent tank 44 or recirculated directly to recirculation system 30 or indirectly to recirculation system 30 via an accessory circuit such as aeration system 50. At the same time, nutrient media is supplied to bed 42 from feed tank 21 by way of recirculation system 30 through a series of input ports 37. As seen in FIG. 1, input ports 37a-i are spread throughout microorganism bed 42. Sequential controllers, such as timers (not shown) are operatively connected to enable each input port 37 to discharge fresh nutrient media and/or recirculated nutrient media into a local or small area of bed 42. Sequential controllers are defined as one or more devices operatively connected to one or more discharge ports 37 to control the sequence in which the intake ports open to receive the nutrient medium under pressure. By operatively connected is meant that the timers or other operatively connected sequential controllers are connected either directly or indirectly to one or more components of system 10 so as to control the action of the connected component. For example, timers may be operatively connected to open one of intake ports 37 at a particular time and hold it open for a specific length of time. In an alternate embodiment, one timer may be operatively connected to more than one intake port 37. In one embodiment, a sequential controller may be a programmable microcontroller. At discharge of one of the plurality of input ports 37, a mixture of nutrient-media and microorganisms, localized around the discharging port 37, is stirred by the pressure of the quantum of discharged nutrient media (“plug”) and mixed together to feed the microorganisms, while at the same time the remainder of the bed is quiescent enabling the release of desired product that is relatively free of media and microorganisms. The overall quiescence of bed 42 also enables microorganisms stirred by the discharge plug to fall back into the bed. After mixing, the mixed segment of microorganisms is allowed to settle. Settling times can range from 10 seconds to 30 minutes.

Overall bed quiescence is maintained by discharging intake ports 37 one at a time. In a preferred embodiment, adjacent input ports 37 are not discharged successively. This allows the areas of bed 42 around a plug to remain as unmoving as possible and enables the portion of bed 42 that is mixing to become dormant as soon as possible. Therefore, after one input port 37a discharges, the next one to discharge will preferably be some distance from the preceding discharge port to keep some distance between the mixing plugs formed by the nutrient media discharged into bed 42. In an alternate embodiment, two or more nonadjacent intake ports 37 may discharge simultaneously into reactor 41.

As the microbes of bed 42 produce the desired product, that product separates from the bed and nutrient media as it rises (or falls) within reactor 41 and forms layer 49. In the example shown in FIG. 1, the product is lighter than the nutrient media and floats to the top of the reactor to form layer 46. As fresh nutrient media is discharged into reactor 41, older nutrient media is also pushed up toward layer 46 by the fresher media. As this upward movement proceeds, the lighter product produced by bed 42 continues to separate from the rising nutrient media to form a more concentrated portion of layer 46. Product is then drawn through outlet 43 into effluent tank 44 where it is either stored or processed further. Sample port 45 is positioned between reactor 41 and tank 44 to allow product samples to be taken for quality control and other purposes.

Nutrient media that collects in a different portion of layer 46 is removed from reactor 41 through nutrient outlet 48. Nutrient media may be diverted directly to recirculation system 30 through pipe 48a or it may be carried to an accessory circuit for further processing or renewal. An example of such an accessory circuit is aeration circuit 50.

Recirculation circuit 30 allows a consistent pressure for the discharge of nutrient media though input ports 37a-i to create the necessary mixing of the nutrient media with the microorganisms within bed 42. It allows the necessary pressure to be applied without the simultaneous introduction of introducing new nutrient media from media input system 20 leading to a more efficient use of the nutrient media. In addition to being connected to reactor 41 through recirculation system 30, it will be recognized that media input system 20 may be directly connected to intake ports 37 to feed fresh nutrient media into reactor 41 with each discharge into reactor 41. Also, in an alternate embodiment not shown here, a bypass line may constructed between media input system 20 and reactor 41 to bypass recirculation system 30. In such an embodiment, input pump 27 would pump directly to intake ports 37a-i.

If adjustments to the nutrient media are needed, they can be introduced through recirculation system 30. As can be seen in FIG. 1, nutrient input system 20 is connected to recirculation system 30. Fresh nutrient media is introduced to bed 42 by way of recirculation system 30. In addition, if adjustments to the media, such as, for example, O₂ levels or pH, are necessary, these modifications can be introduced through recirculation system 30 without interruption of the operation of system 10 such as the introduction of media into reactor 41.

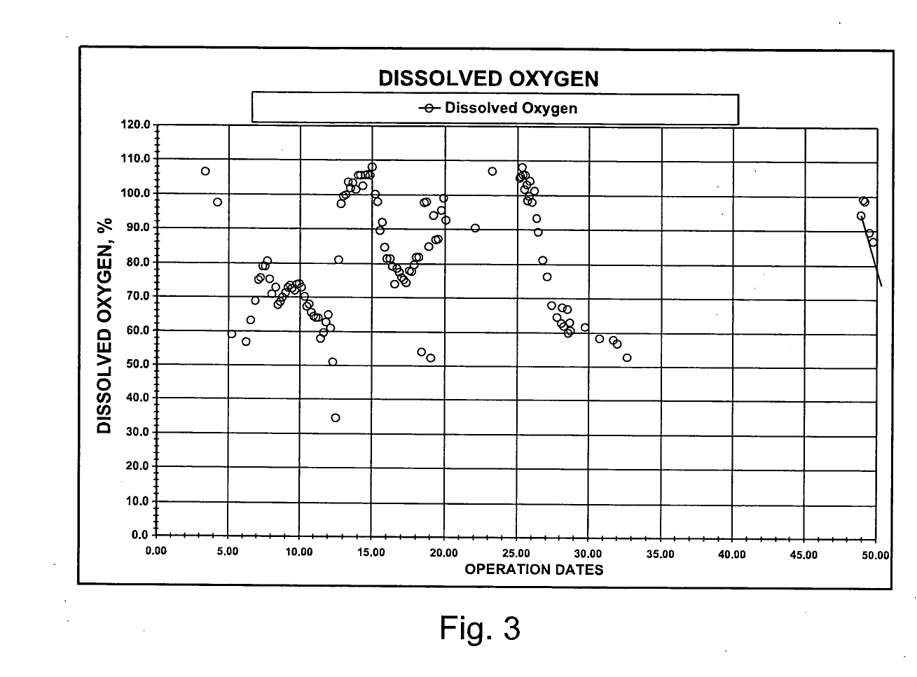

For example, aeration system 50 is connected to recirculation system 30 by means of line 54. Nutrient media removed from reactor 41 through line 48 is received into aeration chamber 51. Aeration chamber 51 also receives oxygen from tank 52 through oxygen line 53 with oxygen exiting at the bottom of chamber 51. Aeration stones 53a cause the exiting oxygen to bubble up into the nutrient media thereby increasing the O₂ levels in the nutrient media. The reoxygenated media is then input back into reactor 41 through recirculation system 30. It will be recognized by those skilled in the art that more than one accessory system or circuit 50 may be included in system 10. In addition, it will be recognized that more than one recirculation system may be incorporated into system 10. See FIG. 3 showing the changing levels of dissolved oxygen in the nutrient media throughout the inositol production run discussed below.

If necessary, gas collection system 60 is connected to reactor tank 41. As is well known by those skilled in the art, production of fermentation products often includes the production of gases such as carbon dioxide. Gas collection system 60 enables a user to capture gas product(s) for testing or further refinement. Gas collector 61 is connected to reactor tank 41 by gas outlet pipe 47 to divert gases from reactor 41. Flow into gas collector 61 is controlled by valve 64. Pipe 67 extends from pipe 47 and leads to gas meter 62 to measure flow. Also seen within pipe 67 is valve 63 and gas sampler 65.

Persons of skill in the art will recognize that automatic sensing and regulating devices may be incorporated into system 10 to provide information to the user regarding parameters such as glucose quantities, O₂ concentrations, and ethanol levels. These may be positioned at appropriate locations in system 10. For example, sensors 72, 73, and 74 are positioned in line 48 carrying nutrient media from reactor 41. Information from these sensors and gauges may be used to determine the adjustments and modifications to be made into the media as well as enabling the user to decide when fresh media is to be introduced into recirculation system 30 from input system 20. These modifications allow the user to maintain bed 42 in a steady state condition. It will be recognized that sensors 72, 73, and 74, as well as other similar devices, may be automated and controlled by a programmable microcontroller.

The microorganisms of bed 42 are preferably supported on inert particles which increase the amount of surface area available for support of the microorganisms. Preferably, the support particle is as small as possible, preferably below 150 microns in width or diameter to provide an adequate surface for microbial attachment yet still possess a specific gravity which will be greater than 1.0 if the bed is desired to sink to the bottom of reactor 41 or less than 1.0 if bed 42 is desired to float in reactor 41. For example, if the desired product is heavier than water, which has a specific gravity of 1.0, it would be advantageous for bed 42 to float to separate the microorganisms and particles from the product. In this case, product layer 46 is located toward the bottom of reactor 41.

In one embodiment, silica is used to support the microorganisms. Other materials include, but are not limited to, calcium alginate beads, diatomaceous earth, activated carbon, crystalline cellulose, activated alumina, filter paper, polystyrene beads, and vermiculate. Persons of skill in the art will recognize that the actual support material(s) used for a particular reaction may depend on the microorganisms to be used in the fermentation reaction and compatibility with the nutrient media supplied to bed 42.

Although system 10 is described above for production of a product lighter than the nutrient media, it will be recognized that system 10 may be used for production of a product heavier than the nutrient media. In this situation, fresh and/or recirculated media may be introduced into the top of reactor 41 again in a series of pulses such that the plug of nutrient and stirred microorganisms is directed downward into bed 42. In this embodiment, the microorganisms of bed 42 may agglutinate to forms clumps of groups of massed microorganisms rather than be attached to support particles. These agglutinated clumps may be used to replace support particles. The use of agglutinated microorganisms may be advantageous when a floating bed 42 is desired, as with production of a product with a specific gravity greater than 1.0.

### EXAMPLE

Forms of recombinant yeasts designed to produce inositol are described in U.S. Pat. Nos. 5,529,912 and 5,599,701, both to Henry and White and U.S. Pat. No. 6,645,767 to Villa and White, which patents are hereby incorporated by reference in their entirety. As described in the incorporated references, cultures of the recombinant Saccharomyces cerevisiae (S. cerevisiae) yeast were genetically altered to disable the encoding to a negative regulator of phospholipid biosynthesis, as well as possess both multiple copies of an INO1 gene and an OP11 gene deletion which results in deregulation of inositol production. The recombinant species produces ethanol and inositol in a low ethanol environment necessitating the continuous removal of ethanol during the inositol production run.

To produce inositol using system 10, a starter culture of the above modified S. cerevisiae was combined with 100 grams of yeast extract into a nutrient media comprising 200 grams of peptone and 50 grams of glucose added to tap water to produce 10 liters of solution. The solution was sterilized by autoclaving and input from feed tank 21 to reactor 41 at a rate of 0-5 ml/min. Reactor 41 volume was 5 liters. Oxygen was added to the nutrient media by outputting the media from reactor 41 to aeration chamber 51 where oxygen was added to the media. The oxygenated media was then recycled to reactor 41 through recirculation system 30. Oxygen was maintained in chamber 51 above 50 mg/l and adjusted to a minimum of 5 mg/l in reactor 41.

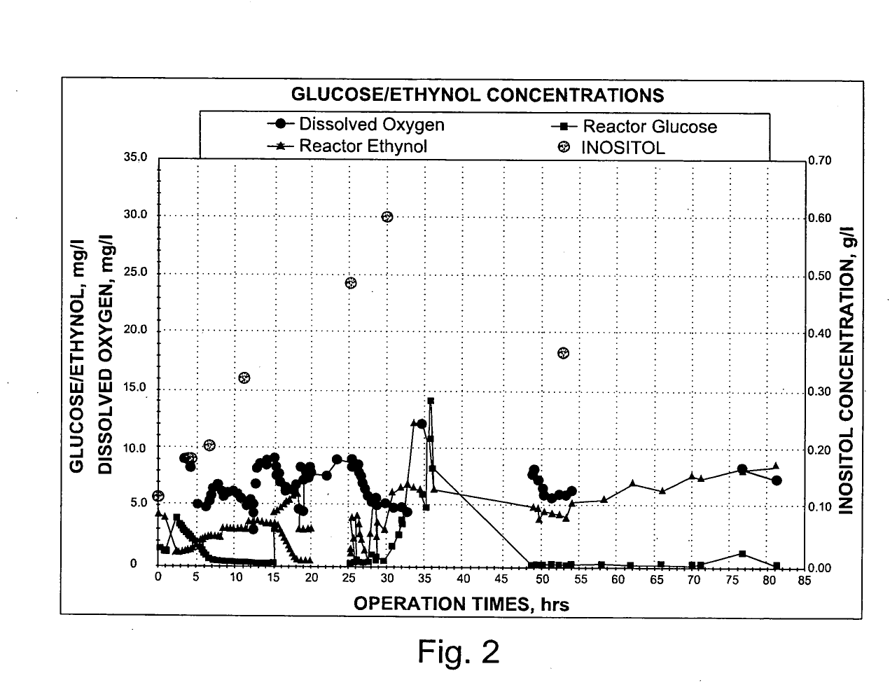

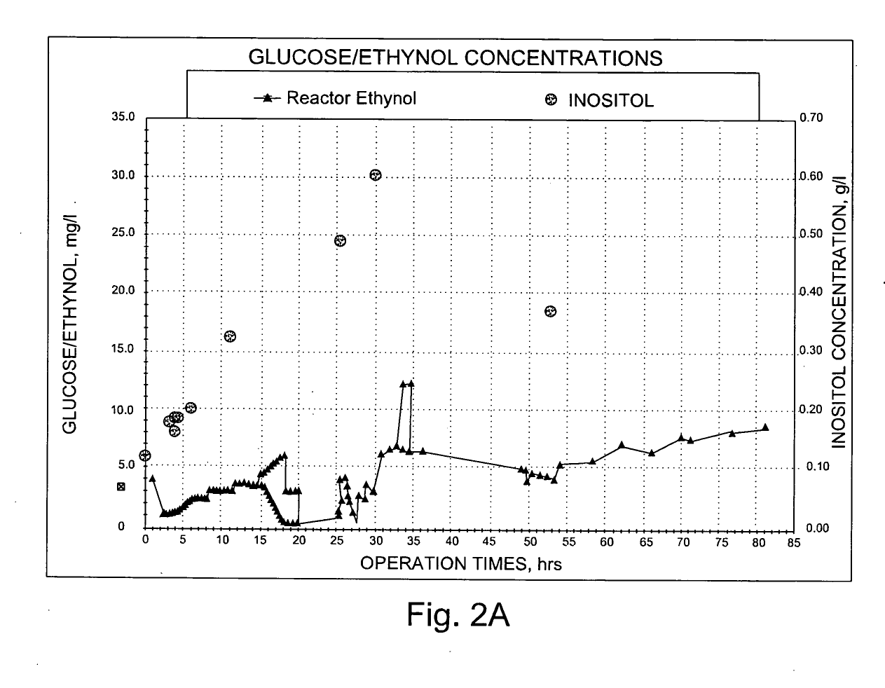

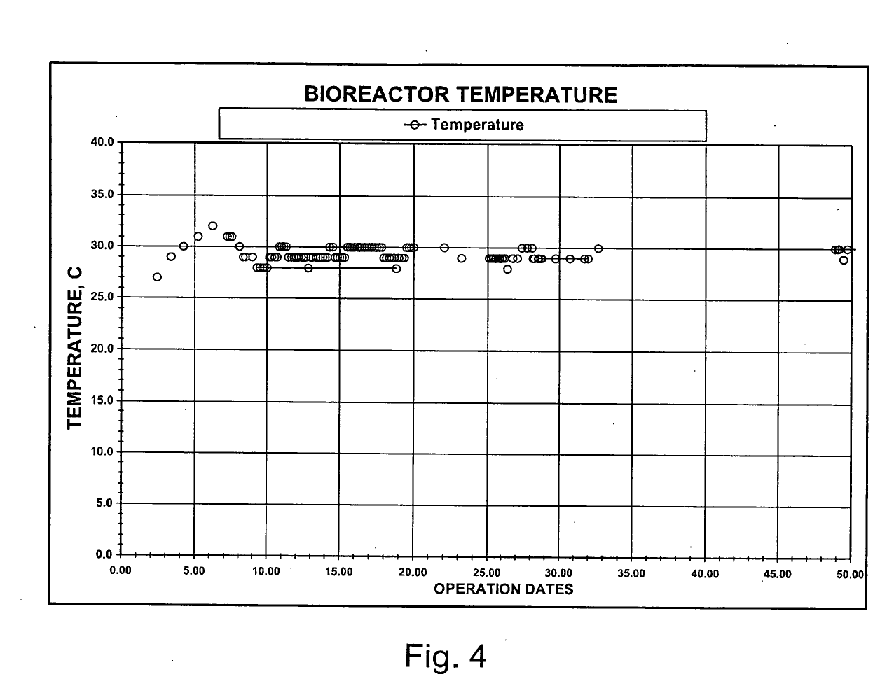

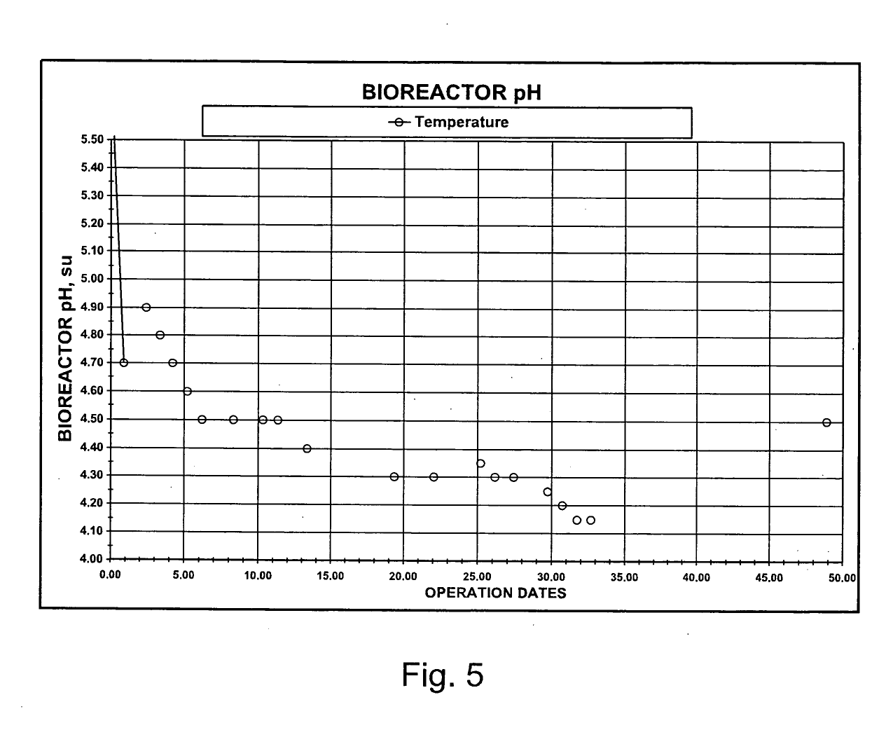

After approximately nine hours, reactor 41 was determined to be in a steady state condition. Concentrations of glucose sampled from line 48 ranged from almost zero to 14.2 g/l with an average range between 0.0 and 5 g/l. As discussed above, ethanol was produced with inositol and floated to layer 46 within reactor 41. To maintain inositol production, ethanol was removed through recirculation line 48. As can be seen from FIGS. 2 and 2A, the recirculated material contained ethanol ranging in concentration from slightly less than 1 to about 7 g/l. This consistent range indicated that the microorganisms of bed 42 were being maintained in a steady state condition. This is also seen in FIGS. 3-5 showing that DO (50-110%), temperature (˜30° C.) of reactor 41, and pH (4-5) all remained within acceptable parameters during the duration of the inositol fermentation process. See FIGS. 3-5 showing the readings for these parameters. Nutrient media was input into reactor 41 through input ports at periodic intervals and for varying lengths of time to maintain the steady state condition and bed 42.

Because inositol is not readily measured on a continuous basis, inositol production was indirectly measured by quantifying ethanol output as it was discovered that the modified S. cerevisiae produce inositol and ethanol simultaneously when ethanol is present in low quantities. In addition, samples of inositol were taken at intervals throughout the production run for later measurement. However, if the concentration of ethanol in the media surrounding the microorganisms becomes too high the production of inositol is reduced or stopped entirely. FIGS. 2 and 2A shows the results of the periodic sampling and quantifying of inositol. At time zero (after the steady state was established), the first sample was measured at about 0.12 g/l. Inositol production is seen to increase in a linear fashion from 0.12-0.6 g/l at 30 hours of continuous steady state operation. During this initial 30 hour period (0-30 hours in FIG. 2A), ethanol concentration ranged from about 0.2 mg/l to about 7.0 mg/l—that is within the steady state parameters. However, at 33 hours, there was an increase in ethanol concentration to about 12 mg/l followed by a drop to 7.0 mg/l over the next 4 hours. By 49 hours (about 15 hours after the ethanol peak), ethanol concentration was measured at 5.0 mg/l and inositol concentration measured dropped to about 0.37 g/l. This drop in inositol production concurrent with an increase in ethanol concentration in the nutrient media demonstrates the necessity of continuous removal of ethanol to maintain a low (0-7 mg/l) concentration of ethanol in the nutrient media. However, inositol measurements did not fall to the initial steady state levels of 0.12 g/l or below following the ethanol peak. Instead, the system recovered to continue producing inositol as evidenced by the 0.37 g/l reading at 53 hours.

### Hypothetical Example

System 10 is used to produce penicillin in a continuous operation. P. chrysogenum spores are maintained in a sporulation media comprising malt extract 20, glucose 20, and peptone 1.0 with a pH range of 6.5±0.2. (Unless otherwise stated, all units are in g/L.). To induce spore germination, the spores are placed in a growth media comprising glycerol, 7.5, peptone 5.0, molasses 7.5, MgSO₄ 0.05, KH₂PO₄ and NaCl 4.0 for 5 days at 27° C. At the start of germination, spores are introduced to support media to allow the germinating cells to grow on the support media particles to increase overall cell surface area exposed to the media. Germinated P. chrysogenum are maintained on support particles during the production phase described below. An example of support particles is, but not limited to, celite.

To produce penicillin, a culture of Penicillium chrysogenum (P. chrysogenum) is incubated and brought to a steady state condition in reactor 41 after approximately 120 hours. Production media is continuously introduced into reactor 41 through ports 37a-i in the manner described above, that is a single “plug” of media is discharged into reactor 41 through a single intake port 37. The discharge pressure causes the portion of the bed in proximity to the discharging port 37 that contains the germinated P. chrysogenum cells to mix with the incoming growth media and causing the cells to continue producing penicillin. Because the remainder of the bed is not in proximity to the discharging port, it remains quiescent. This quiescence enables the produced penicillin to rise to production layer 49 and to be drawn from the reactor in a manner similar to the removal of inositol described above. The production medium comprises lactose monohydrate 0.13, phenyl acetic acid 0.004, and ammonia 0.4 ml/L with a pH of 6.

Thus it is seen that the objects of the invention are efficiently obtained, although changes and modifications to the invention should be readily apparent to those having ordinary skill in the art, which changes would not depart from the spirit and scope of the invention as claimed.

I claim:

1. A process for the continuous production of a fermentation product comprising:
establishing a steady state condition for a colony of a fermentation microorganism on a bed of support particles in a production reactor;
discharging a quantity of nutrient medium into said production reactor sequentially one at a time through at least two nonadjacent intake ports of a plurality of intake ports thereby mixing said nutrient medium with a segment of said colony;
allowing said segment to settle into said bed of support particles for a predetermined settling time;
separating said fermentation product from said nutrient medium;
withdrawing said nutrient medium from said production reactor;
recirculating said nutrient medium into said fermentation colony; and,
repeating said discharging through said recirculation steps as needed.

2. The process as recited in claim 1 wherein said colony of fermentation microorganisms is genetically modified to produce one or more fermentation products.

3. The process as recited in claim 1 further comprising aerating said recirculated nutrient medium.

4. The process as recited in claim 1 further comprising adding more of said nutrient medium into said recirculated nutrient medium for introduction into said colony of fermentation organisms.

5. The process as recited in claim 1 further comprising diverting gaseous products produced by said colony from said production reactor.

6. The process as recited in claim 1 wherein said predetermined settling time ranges from 10 seconds to 30 minutes.

7. The process as recited in claim 1 wherein said nutrient medium and said colony is maintained at about 30° C. and a pH of about 4-5.

8. The process as recited in claim 1 wherein said fermentation colony comprises Saccharomyces cerevisiae (S. cerevisiae).

9. The process as recited in claim 8 wherein said fermentation product is ethanol.

10. The process as recited in claim 8 wherein said S. cerevisiae colony is genetically modified to produce inositol.

11. The process as recited in claim 1 wherein said fermentation colony comprises Penicillium chrysogenum (P. chrysogenum).

12. An apparatus for the continuous production of fermentation products comprising:
a fermentation reactor having a bed of support particles supporting a colony of a fermentation microorganism;
a nutrient medium tank;
a plurality of nutrient medium discharge pipes operatively connected between said fermentation reactor and said nutrient medium tank;
an input pump operatively arranged to pump said nutrient medium between said nutrient medium tank and said fermentation reactor through said plurality of nutrient medium discharge pipes;
a sequential controller operatively connected between said plurality of said nutrient medium discharge pipes and said input pump for controlling the input of said nutrient medium through each of said plurality of nutrient medium discharge pipes in sequence; and,
a fermentation product tank connected to said fermentation rector and arranged to receive said fermentation product.

13. The apparatus as recited in claim 12 further comprising a recirculation system, said recirculation system including at least one recirculation pump connected between an output from said fermentation reactor and an input to said fermentation reactor

14. The apparatus as recited in claim 13 further comprising an aeration chamber connected between said output of said fermentation reactor and said at least one recirculation pump.

15. The apparatus as recited in claim 14 further comprising an oxygen source connected to said aeration chamber.

16. The apparatus as recited in claim 13 wherein said nutrient medium is pumped into said recirculation system and is supplied to said plurality of discharge pipes from said recirculation system.

17. The apparatus as recited in claim 12 further comprising a gas collector operatively arranged to collect gas from said fermentation reactor.

18. The apparatus as recited in claim 12 wherein said support particles comprise at least one of silica, calcium alginate beads, diatomaceous earth, activated carbon, crystalline cellulose, activated alumina, filter paper, polystyrene beads, and vermiculate.

19. The apparatus as recited in claim 12 wherein said fermentation microorganisms agglutinate to form clumps, said clumps replacing said support particles.

20. The apparatus as recited in claim 19 wherein said agglutinated fermentation microorganisms replace said support particles.

21. A fermentation product produced by the steps comprising:
establishing a steady state condition for a colony of a fermentation microorganism on a bed of support particles in a production reactor;
discharging a quantity of nutrient medium into said production reactor sequentially one at a time through at least two nonadjacent intake ports of a plurality of intake ports thereby mixing said nutrient medium with a segment of said colony;
allowing said segment to settle into said bed of support particles for a predetermined settling time;
separating said fermentation product from said nutrient medium;
withdrawing said nutrient medium from said production reactor;
recirculating said nutrient medium into said fermentation colony; and,
repeating steps 2-6 as needed.

22. The product as recited in claim 21 wherein said colony of fermentation microorganisms is genetically modified to produce one or more of said fermentation products.

23. The product as recited in claim 21 further comprising the step of aerating said recirculated nutrient medium.

24. The product as recited in claim 21 further comprising the step of adding more of said nutrient medium into said recirculated nutrient medium for introduction into said colony of fermentation organisms.

25. The product as recited in claim 21 further comprising the step of diverting gaseous products produced by said colony from said production reactor.

26. The product as recited in claim 21 wherein said predetermined settling time ranges from 10 seconds to 30 minutes.

27. The product as recited in claim 1 wherein said nutrient medium and said colony is maintained at about 30° C. and a pH of about 4-5.

28. The product as recited in claim 21 wherein said fermentation colony comprises Saccharomyces cerevisiae (S. cerevisiae).

29. The product as recited in claim 28 wherein said fermentation product is ethanol.

30. The product as recited in claim 28 wherein said S. cerevisiae colony is genetically modified to produce inositol.

31. The product as recited in claim 21 wherein said fermentation colony comprises Penicillium chrysogenum (P. chrysogenum).

32. The product as recited in claim 31 wherein said fermentation product is penicillin.
